(19) United States
(12) Patent Application Publication
(10) Pub. No.: US 2023/0303466 A1
(43) Pub. Date: Sep. 28, 2023

(54) OXIDATIVE DEHYDROGENATION PROCESS

(71) Applicant: NOVA CHEMICALS (INTERNATIONAL) S.A., Fribourg (CH)

(72) Inventors: Shahin GOODARZNIA, Calgary (CA); Vasily SIMANZHENKOV, Calgary (CA); Bolaji OLAYIWOLA, Calgary (CA); David Gent, Red Deer (CA); Jeffrey KLUTHE, Lacombe (CA)

(21) Appl. No.: 18/020,480

(22) PCT Filed: Aug. 11, 2021
(86) PCT No.: PCT/IB2021/057406

§ 371 Date: Feb. 9, 2023

Related U.S. Application Data
(60) Provisional application No. 63/064,967, filed on Aug. 13, 2020.

(51) Int. Cl. C07C 5/48; B01J 4/00

(52) U.S. Cl. CPC C07C 5/48; B01J 4/008; B01J 2204/007

(57) ABSTRACT

Embodiments described in examples herein provide methods and systems for increasing a yield from an oxidative dehydrogenation (ODH) reactor. An exemplary method includes controlling a temperature of a feed gas composition at less than 250° C. The feed gas composition is flowed through a feed preheater to form a heated feed gas, wherein in the feed preheater the feed gas composition is heated to between 150° C. and 250° C. The heated feed gas is flowed into the ODH reactor less than 15 seconds after leaving the feed preheater.

### TECHNICAL FIELD

The present disclosure relates generally to oxidative dehydrogenation (ODH) of lower alkanes to form ethylene. More specifically, a feed preheater is provided that brings the temperature of the reactants to reaction temperature, while minimizing side reactions.

### BACKGROUND ART

Olefins like ethylene, propylene, and butylene, are basic building blocks for a variety of commercially valuable polymers. Since naturally occurring sources of olefins do not exist in commercial quantities, polymer producers rely on methods for converting the more abundant lower alkanes into olefins. The method of choice for today's commercial scale producers is steam cracking, a highly endothermic process where steam-diluted alkanes are subjected very briefly to a temperature of at least 800° C. The fuel demand to produce the required temperatures and the need for equipment that can withstand that temperature add significantly to the overall cost. Also, the high temperature promotes the formation of coke which accumulates within the system, resulting in the need for costly periodic reactor shut-down for maintenance and coke removal.

Oxidative dehydrogenation (ODH) is an alternative to steam cracking that is exothermic and produces little or no coke. In ODH, a lower alkane, such as ethane, is mixed with oxygen in the presence of a catalyst and optionally an inert diluent, such as carbon dioxide or nitrogen or steam, to produce the corresponding alkene, along with various other oxidation products may also be produced in this process. Research into process conditions has continued to increase the yield of ethylene from ODH.

### SUMMARY OF INVENTION

An embodiment described herein provides a method for increasing a yield from an oxidative dehydrogenation (ODH) reactor. The method includes controlling a temperature of a feed gas composition at less than 250° C. The feed gas composition is flowed through a feed preheater to form a heated feed gas, wherein in the feed preheater the feed gas composition is heated to between 150° C. and 250° C. The heated feed gas is flowed into an ODH reactor less than 15 seconds after leaving the feed preheater.

Another embodiment described herein provides a feed preheater for an ODH reactor, wherein the feed preheater is configured to heat a reactor feed to between about 150° C. and about 250° C. and flow the reactor feed into the ODH reactor within 15 seconds of the reactor feed reaching a maximum temperature.

Another embodiment described herein provides an oxidative dehydrogenation (ODH) reactor including a feed preheater, wherein the feed preheater is configured to heat a reactor feed to between about 150° C. and about 250° C., and to introduce the reactor feed into the ODH reactor within less than 15 seconds of the reactor feed reaching a maximum temperature.

In an aspect, the ODH reactor includes a feed tube disposed within the ODH reactor, wherein the feed tube is heated by contents of the ODH reactor, and a length of the feed tube is selected to heat the reactor feed to between about 150° C. and about 250° C. In an aspect, the ODH reactor includes a second feed preheater for heating a second reactor feed to between about 150° C. and about 250° C., and to introduce the second reactor feed into the ODH reactor within less than 15 seconds of the reactor feed reaching a maximum temperature. In an aspect, the second reactor feed includes acetic acid, ethanol, or a recycle stream, or any combinations thereof.

### BRIEF DESCRIPTION OF DRAWINGS

FIGS. 1A and 1B are block diagrams of an example oxidative dehydrogenation system for the oxidative dehydrogenation of light hydrocarbons.

FIGS. 2A and 2B are schematic drawings of an example chemical complex, showing a preheater for a reactor feed.

FIGS. 3A and 3B are schematic drawings of an example chemical complex, showing a preheater for a mixed reactor feed.

FIG. 4 is a schematic of an example experimental reactor unit.

FIG. 5 is a simplified block flow diagram of a scale-up reactor system used for larger scale tests.

FIG. 6 is a plot of the operating results for a first example using the scale-up reactor.

FIG. 7 is a plot of the results of a second example using the scale-up reactor.

FIG. 8 is a process flow diagram of a method for increasing the yield from an ODH reactor while decreasing side products and fouling upstream of the ODH reactor.

### DESCRIPTION OF EMBODIMENTS

Oxidative dehydrogenation (ODH) reactors use a selective oxidation process to form ethylene, or other alpha-olefins, from ethane. In ODH reaction systems, the feed is heated to reaction temperatures in upstream equipment prior to reaching the reactor, which prevents thermal shock from damaging the catalyst in the reactor. However, as the feed is conveyed to the reactor, reactions in the gas or catalyzed by the walls of the piping may occur, for example, forming acetic acid and solid fouling on the surfaces of the piping. The presence of these thermal reactions prior to an ODH reactor will negatively impact overall plant economics and operational reliability.

As discussed with to respect to the examples herein, it has been determined that these reactions are substantially decreased as the temperature of the feed stream is reduced to below 250° C. Accordingly, when the feed reaches a temperature of 250° C. or greater, it should have a short residence time, such as less than about 13 seconds, before being fed to the reactor. As used herein, the residence time is determined by the internal volume of the preheater vessel divided by the volumetric flow rate of the feed gases at standard temperature and pressure (STP) conditions entering the preheater vessel. The STP conditions are 21° C. and 100 kPa.

Embodiments described herein provide a feed preheater for increasing yield from an oxidative dehydrogenation reactor, for example, before the ODH reactor or in the first portion of the ODH reactor. The feed preheater may increase the temperature of a feed to between about 150° C. and about 250° C. and introduce the feed into the reactor within about 15 seconds or less of the feed reaching a target temperature, leaving the feed preheater, or both.

The placement of the feed preheater having a short residence time immediately upstream of an ODH reactor will improve plant economics by decreasing or eliminating unwanted gas phase reactions or reactions catalyzed by the walls of the piping in or after the preheater. It will also improve the operational reliability of plants by decreasing or eliminating fouling upstream of the reactor and by eliminating the potential for process upsets caused by the feed stream entering into a flammable composition envelope.

Other than in the operating examples or where otherwise indicated, all numbers or expressions referring to quantities of ingredients, reaction conditions, etc. used in the specification and claims are to be understood as modified in all instances by the term “about”. Accordingly, unless indicated to the contrary, the numerical parameters set forth in the following specification and attached claims are approximations that can vary depending upon the desired properties, which the present disclosure desires to obtain. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, the term “alkane” refers to an acyclic saturated hydrocarbon. In many cases, an alkane consists of hydrogen and carbon atoms arranged in a linear structure in which all of the carbon-carbon bonds are single bonds. Alkanes have the general chemical formula CnH2n+2. In many embodiments of the disclosure, alkane refers to one or more of methane, ethane, propane, butane, pentane, hexane, heptane, octane, nonane, decane and dodecane. In particular embodiments, alkane refers to ethane and propane.

As used herein, the term “alkene” refers to unsaturated hydrocarbons that contain at least one carbon-carbon double bond. In many embodiments, alkene refers to alpha olefins. In many embodiments of the disclosure, alkene refers to one or more of ethylene, propylene, 1-butene, pentene, pentadiene, hexene, octene, decene and dodecene. Further, as used herein, the term includes other compounds with carbon-carbon double bonds, such as butadiene, among others. In particular embodiments, alkene refers to ethylene and propylene and, in some embodiments, ethylene.

As used herein, the terms “alpha olefin” or “α-olefin” refer to a family of organic compounds which are alkenes (also known as olefins) with a chemical formula CₓH₂ₓ, distinguished by having a double bond at the primary or alpha position. In many embodiments of the disclosure, alpha olefin refers to one or more of ethylene, propylene, 1-butene, 1-pentene, 1-hexene, 1-octene, 1-decene and 1-dodecene. In particular embodiments, alpha olefins refer to ethylene and propylene and, in some embodiments, ethylene.

As used herein, the term “essentially free of oxygen” means the amount of oxygen present, if any, remaining in a process stream after the one or more ODH reactors, and in many embodiments after the second reactor as described herein, is low enough that it will not present a flammability or explosive risk to the downstream process streams or equipment. Further, reducing the oxygen content will lower the degradation rate of the amine solution in the downstream amine tower and reduce polarization in the downstream compression stage.

As used herein, the term “fixed bed reactor” refers to one or more reactors, in series or parallel, often including a cylindrical tube filled with catalyst pellets with reactants flowing through the bed and being converted into products. The catalyst in the reactor may have multiple configurations including, but not limited to, one large bed, several horizontal beds, several parallel packed tubes, and multiple beds in their own shells.

As used herein, the term “fluidized bed reactor” refers to one or more reactors, in series or parallel, often including a fluid (gas or liquid) which is passed through a solid granular catalyst, which can be shaped as tiny spheres, at high enough velocities to suspend the solid and cause it to behave as though it were a fluid.

As used herein, an ODH catalyst refers to any catalyst capable of functioning as in an ODH process. For example, a catalyst of the formula “MoVOx” refers to a mixed metal oxide having the empirical formula Mo_{6.5-7.0}V₃O_{d}, where d is a number to at least satisfy the valence of any present metal elements; a mixed metal oxide having the empirical formula Mo_{6.25-7.25}V₃O_{d}, where d is a number to at least satisfy the valence of any present metal elements, or combinations thereof.

It can be noted, however, that any catalyst used for ODH may be used in embodiments described herein, as the choice of catalyst does not affect the operations of the upstream feed preheater. The catalyst materials may include molybdenum (Mo), vanadium (V), oxygen (O), and any number of other elements, including, for example, iron (Fe), aluminum (Al) or beryllium (Be), among others.

As used herein, the term “catalyst material” refers to a material that includes an active catalyst that can promote selective oxidation (SO) reactions, such as the oxidative dehydrogenation of ethane to ethylene, for example, on a support. The catalyst material can be a plurality of particles or a formed catalyst material. Non-limiting examples of formed catalyst materials include extruded catalyst materials, pressed catalyst materials, and cast catalyst materials. Non-limiting examples of pressed and cast catalyst materials includes pellets-such as tablets, ovals, and spherical particles.

As used herein, the term “catalyst” generally refers to the active catalyst portion of a catalyst material. The catalyst is generally processed in further steps to form a catalyst material. The catalyst material may also be processed in further steps to form a final catalyst material.

The preheater system described herein can be used with any number of reaction processes. For example, the preheater may be used with an ODH process as described herein, or in other processes that can benefit from heating feedstocks immediately prior to injecting them into a reactor, avoiding side reactions. In various embodiments, the preheater is used with a steam cracking unit.

### The Oxidative Dehydrogenation (ODH) System

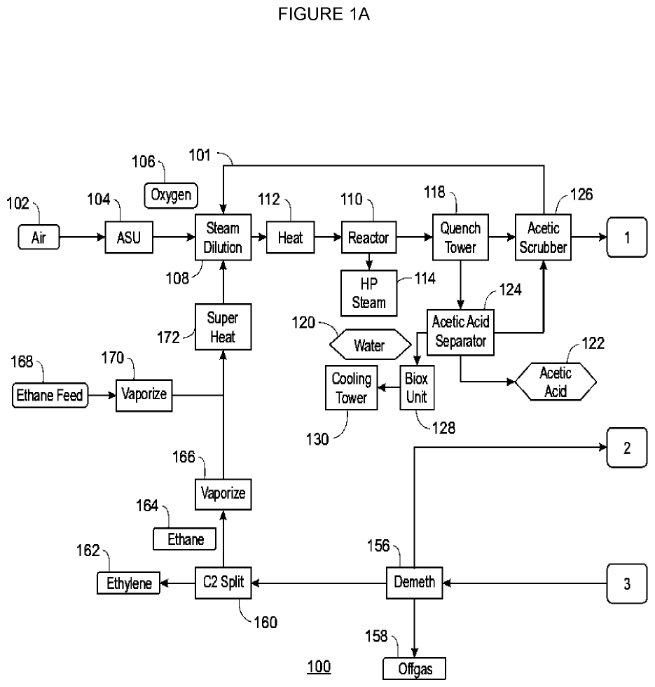

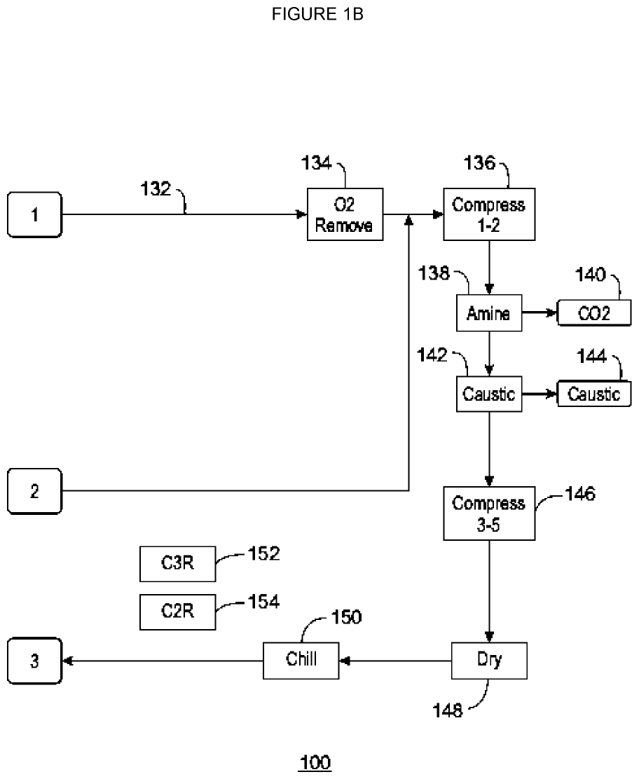

FIGS. 1A and 1B are block diagrams of an oxidative dehydrogenation system 100 for the oxidative dehydrogenation of light hydrocarbons, in accordance with examples. The oxidizing agent generally used in the process is air 102, although oxygen, generally mixed with a diluent, may also be used. The air 102 is flowed into an air separation unit (ASU) 104. In the ASU 104, the oxygen 106 is separated from other gases, such as nitrogen and carbon dioxide, among others. The oxygen 106 may then be mixed with a diluent, for example, in a steam dilution system 108.

To avoid process upsets, in many embodiments, mixtures of one or more alkanes with oxygen are employed using ratios that fall outside of the flammability envelope of the one or more alkanes and oxygen. In some embodiments, the ratio of alkanes to oxygen may fall outside the upper flammability envelope. In these embodiments, the percentage of oxygen in the mixture can be less than 30 vol. %, in some cases less than 25 vol. %, or in other cases less than 20 vol. %, but greater than zero.

In embodiments with higher oxygen percentages, alkane percentages can be adjusted to keep the mixture outside of the flammability envelope. While a person skilled in the art would be able to determine an appropriate ratio level, in many cases the percentage of alkane is less than about 40 vol. % and greater than zero. As a non-limiting example, where the mixture of gases prior to ODH includes 10 vol. % oxygen and 40 vol. % alkane, the balance can be made up with an inert diluent. It can be noted that “inert diluent”, as used herein, refers to the influence of the diluent on flammability, not whether the diluent, such as carbon dioxide or steam, can participate in the ODH reaction. Non-limiting examples of useful inert diluents in this embodiment include, but are not limited to, one or more of steam, nitrogen, and carbon dioxide, among others. In some embodiments, the inert diluent should exist in the gaseous state at the conditions within the reactor and should not increase the flammability of the hydrocarbon added to the reactor, characteristics that a skilled worker would understand when deciding on which inert diluent to employ. The inert diluent can be added to either of the alkane containing gas or the oxygen containing gas or both separately prior to entering the ODH reactor.

Although a number of different hydrocarbons may be used, in an oxidative dehydration process, generally ethane is provided to the reactor along with oxygen. In some embodiments, the volumetric feed ratio of oxygen to ethane (O₂/C₂H₆) provided to the one or more ODH reactors can be at least about 0.3, in some cases at least about 0.4, and in other cases at least about 0.5 and can be up to about 1, in some cases up to about 0.9, in other cases up to about 0.8, in some instances up to about 0.7 and in other instances up to about 0.6. The volumetric feed ratio of oxygen to ethane can be any of the values or range between any of the values recited above.

In some embodiments, mixtures that fall within the flammability envelope may be employed, for example, in instances where the mixture exists in conditions that prevent propagation of an explosive event. In these non-limiting examples, the flammable mixture is created within a medium where ignition is immediately quenched. As a further non-limiting example, a user may design a reactor where oxygen and the one or more alkanes are mixed at a point where they are surrounded by a flame arresting material. Any ignition would be quenched by the surrounding material. Flame arresting materials include, but are not limited to, metallic or ceramic components, such as stainless steel walls or ceramic supports. In some embodiments, oxygen and alkanes can be mixed at a low temperature, where an ignition event would not lead to an explosion, then introduced into the reactor before increasing the temperature. The flammable conditions do not exist until the mixture is surrounded by the flame arrestor material inside of the reactor.

The amount of steam added to the ODH process in the steam dilution system 108 affects the degree to which carbon dioxide acts as an oxidizing agent. In some embodiments, steam may be added directly to the ODH reactor 110, or steam may be added to the individual reactant components—the lower alkane, oxygen, or inert diluent—or combinations thereof, and subsequently introduced into the ODH reactor 110 along with one or more of the reactant components. Alternatively, steam may be added indirectly as water mixed with either the lower alkane, oxygen or inert diluent, or a combination thereof, with the resulting mixture being preheated before entering the reactor.

As described herein, a residence time of the hydrocarbons and oxygen at temperatures above about 250° C., for example, of greater than about 13 seconds, may lead to undesirable reactions that cause the formation of impurities and fouling in the piping from the steam dilution system 108 to the reactor. Accordingly, the temperature of the steam dilution system 108 may be decreased, or water addition may be used, to lower the probability of side reactions. In some embodiments, wet steam is used for in the steam dilution system 108, at a temperature of between about 95° C. and 250° C. When adding lower temperature steam, or adding steam indirectly as water, a heater 112 is used to increase the temperature so that the water is entirely converted to steam before entering the reactor, decreasing the probability of damage to the catalyst. In various embodiments, the heater 112 is configured to raise the temperature to between about 150° C. and about 250° C. within about 15 seconds, or 13 seconds, or less, before the mixed feed is added to the reactor 110.

Increasing the amount of steam, or water, added to the ODH reactor 110 increases the degree to which carbon dioxide acts as an oxidizing agent. Decreasing the amount of steam added to the ODH reactor 110 decreases the degree to which carbon dioxide acts as an oxidizing agent. In some embodiments a user monitors the carbon dioxide output and compares it to a predetermined target carbon dioxide output. If the carbon dioxide output is above the target a user can then increase the amount of steam added to the ODH process. If the carbon dioxide output is below the target a user can decrease the amount of steam added to the ODH process, provided steam has been added. Setting a target carbon dioxide output level is dependent on the requirements for the user. In some embodiments increasing the steam added will have the added effect of increasing the amount of acetic acid and other by-products produced in the process. As larger amounts of acetic acid from the output of the ODH may be generated by higher levels of steam, reducing steam levels will decrease the amount generated. Conversely, higher levels of steam will increase the amount of carbon dioxide consumed.

In some embodiments, the amount of steam added to the ODH reactor 110 can be up to about 75 vol. %. In some circumstances up to about 50 vol. %, in some circumstances up to about 40 vol. %, in some cases up to about 35 vol. %, in other cases up to about 30 vol. %, and in some instances up to about 25 vol. % and can be zero, in some cases at least 0.5 vol. %, in other cases at least 1 vol. %, in other cases at least 5 vol. %, in some instances at least 10 vol. % and in other instances at least 15 vol. % of the stream entering the ODH reactor 110. The amount of steam in the stream entering the ODH reactor 110 can be any value or range between any of the values recited above. As used herein, the ODH reactor 110 may include a single reactor, or multiple reactors.

In some embodiments when using two or more ODH reactors a user may choose to control carbon dioxide output in only one, or less than the whole complement of reactors. For example, a user may opt to maximize carbon dioxide output of an upstream reactor so that the higher level of carbon dioxide can be part of the inert diluent for the subsequent reactor. In that instance, maximizing carbon dioxide output upstream minimizes the amount of inert diluent that would need to be added to the stream prior to the next reactor.

There is no requirement for adding steam to an ODH process, as it is one of many alternatives for the inert diluent. For processes where no steam is added, the carbon dioxide output is maximized under the conditions used with respect to ethane, oxygen and inert diluent inputs. Decreasing the carbon dioxide output can then be a matter of adding steam to the reaction until carbon dioxide output drops to the desired level. In embodiments where oxidative dehydrogenation conditions do not include addition of steam, and the carbon dioxide output is higher than the desired carbon dioxide target level, steam may be introduced into the reactor while keeping relative amounts of the main reactants and inert diluent—lower alkane, oxygen and inert diluent—added to the reactor constant, and monitoring the carbon dioxide output, increasing the amount of steam until carbon dioxide decreases to the target level.

In some embodiments, a carbon dioxide neutral process can be achieved by increasing steam added so that any carbon dioxide produced in the oxidative dehydrogenation process can then be used as an oxidizing agent such that there is no net production of carbon dioxide. Conversely, if a user desires net positive carbon dioxide output then the amount of steam added to the process can be reduced or eliminated to maximize carbon dioxide production. As the carbon dioxide levels increase there is potential to reduce oxygen consumption, as carbon dioxide is competing as an oxidizing agent. The skilled person would understand that using steam to increase the degree to which carbon dioxide acts as an oxidizing agent can impact oxygen consumption. The implication is that a user can optimize reaction conditions with lower oxygen contributions, which may assist in keeping mixtures outside of flammability limits.

In any implementation, lowering the feed temperature until immediately before addition to the ODH reactor 110 will decrease side reactions and the formation of fouling deposits. From the heater 112, the feed is introduced into the ODH reactor 110. The ODH reactor 110 may be any of the known reactor types applicable for an ODH process, such as the ODH of alkanes. In some embodiments, the ODH reactor 110 is a conventional fixed bed reactor. In a typical fixed bed reactor, reactants are introduced into the reactor at one end, and flow past an immobilized catalyst, during which products are formed. The products leave the ODH reactor 110 at the opposite end from where the feed is introduced. Designing a fixed bed reactor suitable for the methods disclosed herein can follow techniques known for reactors of this type.

In some embodiments, the use of inert non-catalytic heat dissipative particles can be used within one or more of the ODH reactors. In various embodiments, the heat dissipative particles are present within the bed and include one or more non catalytic inert particulates having a melting point at least 30° C., in some embodiments at least 250° C., in further embodiments at least 500° C. above the temperature upper control limit for the reaction; a particle size in the range of 0.5 to 75 mm, in some embodiments 0.5 to 15, in further embodiments in the range of 0.5 to 8, in further embodiments in the range of 0.5 to 5 mm; and a thermal conductivity of greater than 30 W/mK (watts/meter Kelvin) within the reaction temperature control limits. In some embodiments the particulates are metal alloys and compounds having a thermal conductivity of greater than 50 W/mK (watts/meter Kelvin) within the reaction temperature control limits. Non-limiting examples of suitable metals that can be used in these embodiments include, but are not limited to, silver, copper, gold, aluminum, steel, stainless steel, molybdenum and tungsten.

The heat dissipative particles can have a particle size of from about 1 mm to about 15 mm. In some embodiments, the particle size can be from about 1 mm to about 8 mm. The heat dissipative particles can be added to the fixed bed in an amount from 5 to 95 wt. %, in some embodiments from 30 to 70 wt. %, in other embodiments from 45 to 60 wt. % based on the entire weight of the fixed bed. The particles are employed to potentially improve cooling homogeneity and reduction of hot spots in the fixed bed by transferring heat directly to the walls of the reactor. As described herein, in embodiments the ODH reactor 110 may be cooled by the generation of high pressure steam 114, for example, in a jacket around or coils within the ODH reactor 110.

Additional embodiments include the use of a fluidized bed reactor, where the catalyst bed can be supported by a porous structure, or a distributor plate, located near a bottom end of the reactor and reactants flow through at a velocity sufficient to fluidize the bed (e.g. the catalyst rises and begins to swirl around in a fluidized manner). The reactants are converted to products upon contact with the fluidized catalyst and the reactants are subsequently removed from the upper end of the reactor. Some embodiments include using a combination of both fixed bed and fluidized bed reactors, each with the same or different ODH catalyst.

In some embodiments, the stream exiting the one or more ODH reactors can be treated to remove or separate water and water soluble hydrocarbons from the stream exiting the one or more ODH reactors. In some embodiments, this stream is fed to a second reactor.

In some embodiments, the stream exiting the ODH reactor 110 is directed to a quench tower 118 to be cooled and condensed. This facilitates the removal of oxygenates, such as water stream 120 and acetic acid stream 122, via a bottom outlet that feeds an acetic acid separator 124. The acetic acid separator 124 separates an acetic acid stream 122 from the water stream 120, as well as separating a gas stream that is returned to an acetic acid scrubber 126. The water stream 120 may be treated in a bio oxidation unit 128 to remove any remaining carbon compounds, such as traces of acetic acid, among others. From the bio oxidation unit 128, the purified water stream 120 may be fed to a cooling tower 130 as a makeup stream.

The remaining gases from the quench tower 118 are fed to the acetic acid scrubber 126, along with separated gases from the acetic acid separator 124. The acetic acid scrubber 126 may remove traces of acetic acid, and other carbon compounds, from these gas streams by oxidation or adsorption.

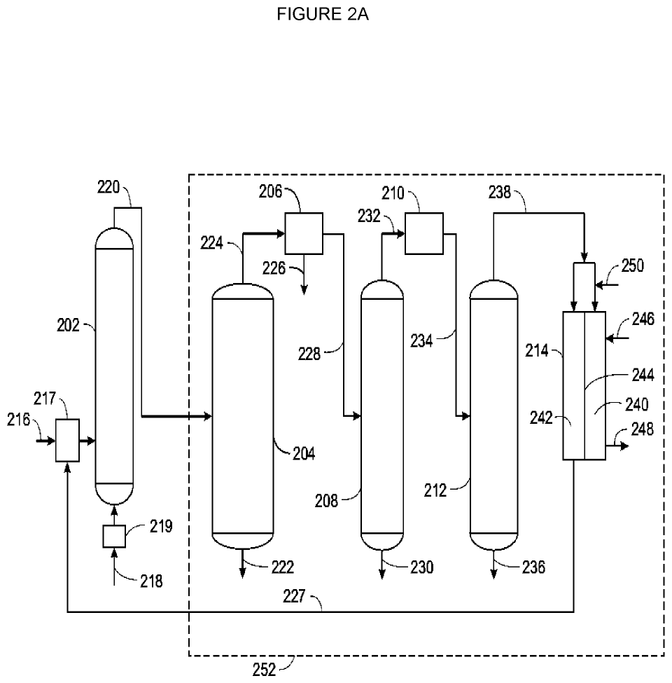

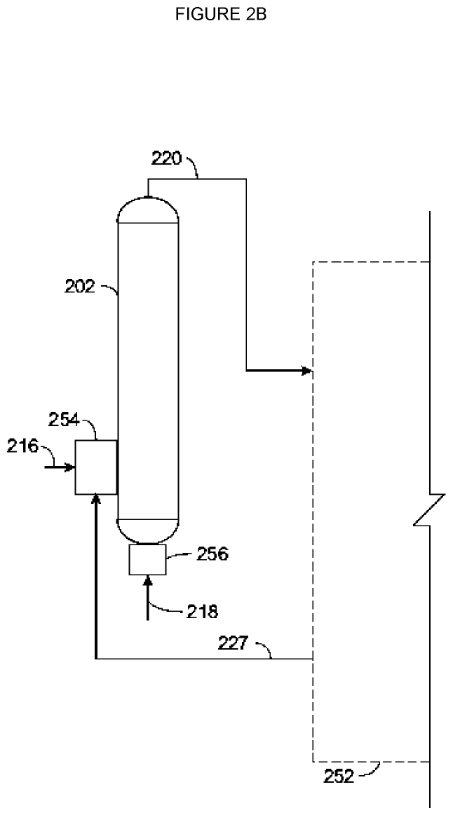

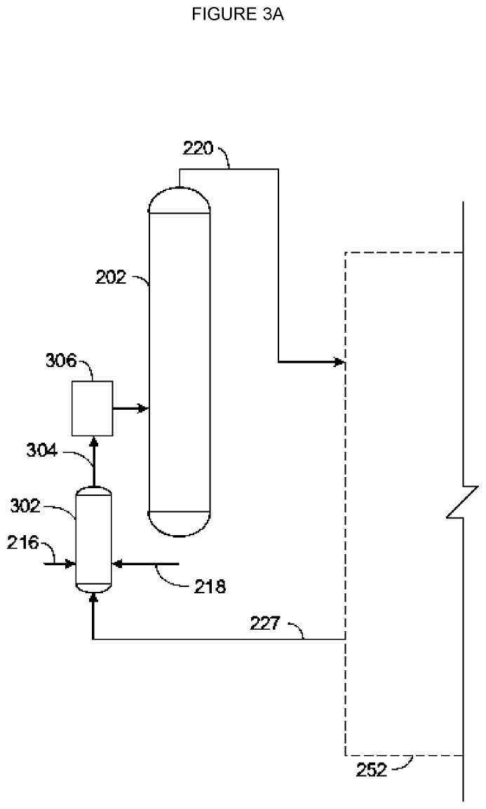

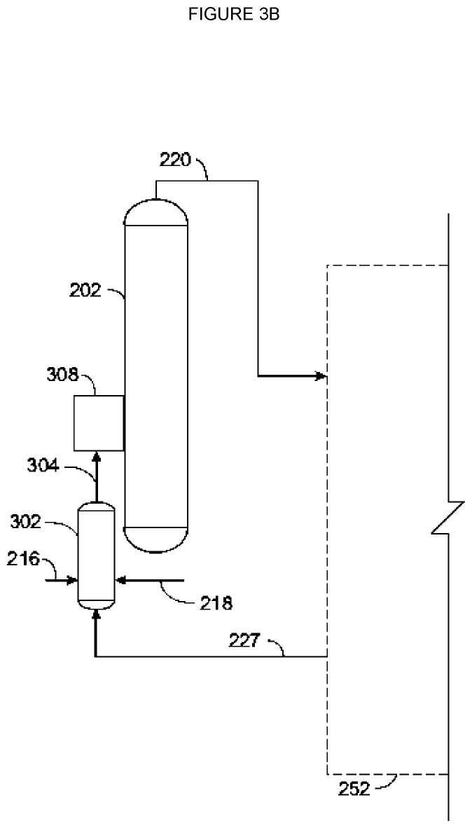

A stream 132 containing unconverted lower alkane (such as ethane), corresponding alkene (such as ethylene), unreacted oxygen, carbon dioxide, carbon monoxide, optionally acetylene and inert diluent, are allowed to exit the acetic acid scrubber 126 and are fed to an oxygen removal system 134 (FIG. 1B), or to a second reactor, as described with respect to FIGS. 2A, 2B, 3A and 3B.

The oxygenates removed via the quench tower 118 and/or acetic acid scrubber 126 can include carboxylic acids (for example acetic acid), aldehydes (for example acetaldehyde) and ketones (for example acetone). The amount of oxygenate compounds remaining in the stream 132 exiting the scrubber and fed to the oxygen removal system 134 will often be zero, for example, below the detection limit for analytical test methods typically used to detect such compounds. When oxygenates can be detected they can be present at a level of up to about 1 per million by volume (ppmv), in some cases up to about 5 ppmv, in other cases less than about 10 ppmv, in some instances up to about 50 ppmv and in other instances up to about 100 ppmv and can be present up to about 2 vol. %, in some cases up to about 1 vol. %, and in other cases up to about 1,000 ppmv. The amount of oxygenates or acetic acid in the stream exiting the scrubber and fed to the oxygen removal system 134 can be any value, or range between any of the values recited above.

In the oxygen removal system 134, as described herein, a high temperature membrane may be used to remove oxygen from the stream 132 exiting the acetic acid scrubber 126. The high temperature membrane may be heated by combusting access hydrocarbons in the stream 132, by combusting fuel added to the oxygen removal system 134, or both. A stream 101 exiting the acetic acid scrubber 126 can be recycled to the steam dilution system 108.

From the oxygen removal system, the stream 132 may be compressed, for example, in a first compressor system 136. The first compressor system 136 may include a single compressor or a series of compressors that sequentially boost the pressure of the stream 132. The compressed stream may then be fed to an amine scrubber 138 to remove CO₂ 140 from the compressed stream, as described in further detail herein. From the amine scrubber 138, the compressed stream may be fed to a caustic wash tower 142. The caustic wash tower 142 further reduces the concentration of CO₂ in the compressed gas stream, sending the CO₂ in a rich caustic stream 144. The rich caustic stream 140 may then be treated to form a lean caustic stream which is returned to the caustic wash tower 138.

The purified gas stream from the caustic wash tower 142 may include unconverted lower alkane (such as ethane) and the corresponding alkene (such as ethylene), and excess inert diluent, such as nitrogen, if used. The purified gas stream may be compressed in a second compressor system 146. The second compressor system 146 may include a single compressor or a chain of compressors that sequentially boost the pressure of the purified gas. The compressed purified gas may then be passed to a dryer 148 to remove excess water vapor from the amine scrubber 138 and the caustic wash tower 142. The dryer 148 may include molecular sieves to adsorb the water, or may include a series of heat exchangers and chillers to physically condense the water, or both.

The dried stream is then passed to a chiller 150. The chiller 150 may include a series of heat exchangers, such as propane chilled heat exchangers, compressed nitrogen chilled heat exchangers, and heat exchangers cooled by fluids from other portions of the process. The chiller 150 may be integrated with, or feed, a depropanizer (C3R) 152, a deethanizer (C2R) 154, or both.

Returning to FIG. 1A, the chilled gas stream is fed to a demethanizer 156. From the demethanizer 156, an off gas stream 158 is sent to waste or to downstream processes. The off gas stream 158 includes the remainder of the inert diluent as well as methane removed from the chilled gas stream. Further, the demethanizer 156 returns a portion of the C₂ compounds, such as ethylene and ethane, to the process upstream of the first compressor system 136. A C₂ stream from the demethanizer 156 is fed to a C₂ splitter 160.

The C₂ splitter 160 divides the C₂ stream into an ethylene product stream 162 and an ethane feed stream 164. The ethane feed stream 164 is vaporized in a heat exchanger 166 to form an ethane gas feed stream. An ethane feed 168 from another ethane source may be vaporized in a heat exchanger 170 and blended into the ethane gas feed stream.

The ethane gas feed stream is then passed through a high temperature heat exchanger 172 to be superheated. In various embodiments described herein, the high temperature heat exchanger 172 heats the ethane gas feed stream to a temperature of less than about 250° C., less than about 220° C., less than about 190° C., or lower. The superheated ethane gas feed stream is then fed to the steam dilution system 108 for use in the process. The core reaction process, including the separation of oxygenates, amine washing, and caustic washing are described further with respect to FIGS. 2 and 3, below.

### The Feed Preheater

FIGS. 2A and 2B are schematic drawings of an example chemical complex, showing a preheater for a reactor feed. FIG. 2A is a schematic diagram of a chemical complex, according to some embodiments. In the following description, like parts are designated by like reference numbers. In some embodiments, the chemical complex includes, in cooperative arrangement, an ODH reactor 202, a quench tower and/or acetic acid scrubber 204, a second reactor 206 (as described herein), an amine wash tower 208, a drier 210, a distillation tower 212, and an oxygen separation module 214. The ODH reactor 202 includes an ODH catalyst capable of catalyzing, in the presence of oxygen which may be introduced via oxygen line 216, the oxidative dehydrogenation of alkanes introduced via alkane line 218. As described herein, an oxidizer preheater 217 is placed on the oxygen line 216 to raise the temperature of the oxidizer feed to reactor temperatures immediately before addition of the oxygen, and any recycled materials, to the reactor 202. Similarly, a hydrocarbon preheater 219 is included to heat the hydrocarbon feed in the alkane line 218 two reaction temperatures before adding the hydrocarbon feed to the ODH reactor 202. Although the second reactor 206 is shown directly after the quench tower or the acetic acid scrubber 204, it can be placed further downstream. In many cases, the process configuration can be more energy efficient if the second reactor 206 is placed after the input stream has been compressed.

The ODH reaction may also occur in the presence of an inert diluent, such as carbon dioxide, nitrogen, or steam, that is added to ensure the mixture of oxygen and hydrocarbon are outside of flammability limits. Determination of whether a mixture is outside of the flammability limits, for the prescribed temperature and pressure, is within the knowledge of the skilled worker. An ODH reaction that occurs within ODH reactor 202 may also produce, depending on the catalyst and the prevailing conditions within ODH reactor 202, a variety of other products which may include carbon dioxide, carbon monoxide, oxygenates, and water. These products leave ODH reactor 202, along with unreacted alkane, corresponding alkene, residual oxygen, carbon monoxide and inert diluent, if added, via ODH reactor product line 220.

ODH reactor product line 220 is directed to quench tower or acetic acid scrubber 204 which quenches the products from ODH reactor product line 220 and facilitates removal of oxygenates and water via quench tower bottom outlet 222. Unconverted lower alkane, corresponding alkene, unreacted oxygen, carbon dioxide, carbon monoxide, and inert diluent added to acetic acid scrubber (quench tower) 204 exit through quench tower overhead line 224 and are directed into second reactor 206.

The temperature of the contents within the ODH reactor product line 220 in a typical ODH process can reach about 450° C. It can be desirable to lower the temperature of the stream before introduction into quench tower or acetic acid scrubber 204 as described above. In that instance, the present disclosure contemplates the use of a heat exchanger immediately downstream of each ODH reactor 202 and immediately upstream of acetic acid scrubber 204. Use of a heat exchanger to lower temperatures in this fashion is well known in the art.

Second reactor 206 contains the group 11 metal with optional promoter and optional support as described above, which causes unreacted oxygen to react with carbon monoxide to form carbon dioxide or, optionally, reacts acetylene to reduce or eliminate it. In second reactor 206, most or all of the unreacted oxygen and acetylene is consumed. All or a portion of the carbon dioxide in second reactor 206 can be recycled back to ODH reactor 202 via recycle lines 226 and 227 to act as an oxidizing agent as described above. The remaining unconverted lower alkane, corresponding alkene, unreacted oxygen (if present), all or part of the carbon dioxide, carbon monoxide (if present), and inert diluent are conveyed to amine wash tower 208 via line 228.

Any carbon dioxide present in line 228 is isolated by amine wash tower 208 and captured via carbon dioxide bottom outlet 230 and may be sold, or, alternatively, may be recycled back to ODH reactor 202 as described above. Constituents introduced into amine wash tower 208 via line 228, other than carbon dioxide, leave amine wash tower 208 through amine wash tower overhead line 232 and are passed through the dryer 210 before being directed to distillation tower 212 through line 234, where C₂/C₂₊ hydrocarbons are isolated and removed via C₂/C₂₊ hydrocarbons bottom outlet 236. The remainder includes mainly C₁ hydrocarbons, including remaining inert diluent and carbon monoxide (if any), which leave distillation tower 212 via overhead stream 238 and is directed to oxygen separation module 214. This stream can also be recycled back to the suction end of the compressor or to boiler for steam superheat.

Oxygen separation module 214 includes a sealed vessel having a retentate side 240 and a permeate side 242, separated by oxygen transport membrane 244. Overhead stream 238 may be directed into either of retentate side 240 or permeate side 242. Optionally, a flow controlling means, as discussed herein, may be included that allows for flow into both sides at varying levels. In that instance an operator may choose what portion of the flow from overhead stream 238 enters retentate side 240 and what portion enters permeate side 242. Depending upon conditions an operator may switch between the two sides, to allow equivalent amounts to enter each side, or bias the amount directed to one of the two sides. Oxygen separation module 214 also includes air input 246 for the introduction of atmospheric air, or other oxygen containing gas, into the retentate side 240. Combustion of products introduced into retentate side 240, due to the introduction of oxygen, may contribute to raising the temperature of oxygen transport membrane 244 to at least about 850° C. so that oxygen can pass from retentate side 240 to permeate side 242. Components within the atmospheric air, or other oxygen containing gas, other than oxygen, cannot pass from retentate side 240 to permeate side 242 and can only leave oxygen separation module 214 via exhaust 248.

As a result of oxygen passing from retentate side 240 to permeate side 242, there is separation of oxygen from atmospheric air, or other oxygen containing gas, introduced into retentate side 240. The result is production of oxygen enriched gas on permeate side 242, which is then directed via oxygen enriched bottom line 227 to ODH reactor 202, either directly or in combination with oxygen line 216 (as shown in FIG. 2A). When overhead stream 238 is directed into retentate side 240 the degree of purity of oxygen in oxygen enriched bottom line 227 can approach 99%. Conversely, when overhead stream 238 is directed into permeate side 242 the degree of purity of oxygen in oxygen enriched bottom line 227 is lower, with an upper limit ranging from 80 vol. %-90 vol. % oxygen, the balance in the form of carbon dioxide, water, and remaining inert diluent, all of which do not affect the ODH reaction as contemplated by the present disclosure and can accompany the enriched oxygen into ODH reactor 202. Water and carbon dioxide can be removed by acetic acid scrubber 204 and amine wash tower 208, respectively. In some embodiments of the disclosure, some or all of the carbon dioxide can be captured for sale as opposed to being flared where it contributes to greenhouse gas emissions. In other embodiments, when carbon dioxide is used in the ODH process, any carbon dioxide captured in the amine wash can be recycled back to ODH reactor 202.

Oxygen transport membrane 244 is temperature dependent, only allowing transport of oxygen when the temperature reaches at least about 850° C. In some embodiments, the components in overhead stream 238 by themselves are not capable, upon combustion in the presence of oxygen, to raise the temperature of oxygen transport membrane 244 to the required level. In this embodiment, the chemical complex of the present disclosure also includes fuel enhancement line 250, upstream of oxygen separation module 214, where combustible fuel, as a non-limiting example methane, may be added to supplement the combustible products from overhead stream 238.

In FIG. 2A, the equipment 252 downstream of the ODH reactor 202 is indicated by a dotted line. This equipment 252 is the same for FIGS. 2B, 3A, and 3B.

FIG. 2B is a schematic diagram of the use of pre-heaters on the feed to and ODH reactor 202. In FIG. 2B, the equipment 252 downstream of the reactor 202 is not shown, but is as described with respect to FIG. 2A. The preheaters 217 and 219 of FIG. 2A are independent devices installed on the piping to the reactor 202.

In some embodiments, the preheaters are constructed against the reactor 202, or in piping in the reactor 202, or both, to take advantage of the heat from the exothermic reaction in the reactor 202. In some embodiments, cooling lines from inside the reactor 202 extend into an oxidizer preheater 254 built against the reactor 202. Similarly, in some embodiments, a hydrocarbon preheater 256 is built against the reactor 202 to allow heat from the reactor 202 to be used for the preheating.

In some embodiments, the preheaters 254 and 256 are built into the reactor 202, for example, as a feed tube in the reactor through which the feeds flow before being introduced into the reactor. In these embodiments, the introduction temperature of the feeds flowing through the preheaters 254 and 256 is controlled by the length of the feed tube, for example, with longer feed tubes bringing the temperature closer to the temperature of the contents of the reactor 202, but adding time to the flow.

In some embodiments, additional preheaters are present, for example, on a feed provided to the ODH reactor from other reactors, termed an interstage feeds The additional preheaters may be used for heating feeds used for the reaction, such as acetic acid, ethanol, and the like. To decrease the amount of side products formed in hot reactor effluents, an effluent cooler may be placed immediately downstream of each reactor to cool the effluent below about 250° C.

FIGS. 3A and 3B are schematic drawings of an example chemical complex, showing a preheater for a mixed reactor feed. FIG. 3A is a schematic diagram of a chemical complex according to some embodiments. In FIG. 3A, the equipment 252 downstream of the reactor 202 is not shown, but is as described with respect to FIG. 2A. A concern in ODH processes is the mixing of a hydrocarbon with oxygen. Under certain conditions the mixture may be unstable and lead to an explosive event. Mixers may be used to mix a hydrocarbon containing gas with an oxygen containing gas in a flooded mixing vessel. By mixing in this way, pockets of unstable compositions are surrounded by a non-flammable liquid so that even if an ignition event occurred it would be quenched immediately. The steam dilution system 108 of FIG. 1A is similar, but uses steam as the inert material. The mixture of gases to the ODH reaction is controlled so that homogeneous mixtures fall outside of the flammability envelope, for the prescribed conditions with respect to temperature and pressure.

As shown in FIG. 3A, in some embodiments, the flooded gas mixer 302 is located upstream of the ODH reactor 202 along the feed lines 216, 218 and 227. In various embodiments, the oxygen line 216 and the alkane line 218 feed directly into the flooded gas mixer 302. As described herein, the temperature of the flooded gas mixer 302 may be limited to below about 250° C. to prevent fouling or other undesirable reactions in the mixed line 304 leaving the flooded gas mixer 302. Accordingly, a homogeneous mixture that includes hydrocarbon and oxygen, and optionally an inert diluent, can be introduced into a preheater 306 from the flooded gas mixer 302 via mixed line 304. In the preheater 306, the temperature of the homogenous mixture is increased over a time span of about 15 seconds, or less, to about 250° C. or less, or between about 150° C. to about 250° C. before the heated homogenous mixture is fed to the reactor 202. At the inlet of the reactor 202, the feed gas could then be heated from less than about 250° C. to the desired reaction temperature. In some embodiments, the feed gas would flow through a short segment of piping in the reactor 202 that includes inert catalyst support balls to increase the temperature before the feed gas comes in contact with the catalyst bed. Oxygen enriched bottom line 227 may feed directly into the flooded gas mixer 302 or in combination with oxygen line 216 into the flooded gas mixer 302.

FIG. 3B is a schematic diagram of a chemical complex according to some embodiments. In FIG. 3B, the equipment 252 downstream of the reactor 202 is not shown, but is as described with respect to FIG. 2A. In various embodiments, a preheater 308 is constructed against the reactor 202, or in piping in the reactor 202, or both, to take advantage of the heat from the exothermic reaction. In some embodiments, cooling lines from inside the reactor 202 extend into the preheater 308 built against the reactor 202.

In some embodiments, the preheater 308 is built into the reactor 202, for example, as a loop of piping through which the feed flows before being introduced into the reactor. In these embodiments, the introduction temperature of the feeds flowing through the preheater 308 is controlled by the length of the loop, for example, with multiple loops bringing the temperature closer to the temperature of the contents of the reactor 202, but adding time to the flow.

The present disclosure also contemplates use of various tools commonly used for chemical reactors, including flowmeters, compressors, valves, and sensors for measuring parameters such as temperature, pressure and flow rates. It is expected that the person of ordinary skill in the art would include these components as deemed necessary for safe operation.

### EXAMPLES

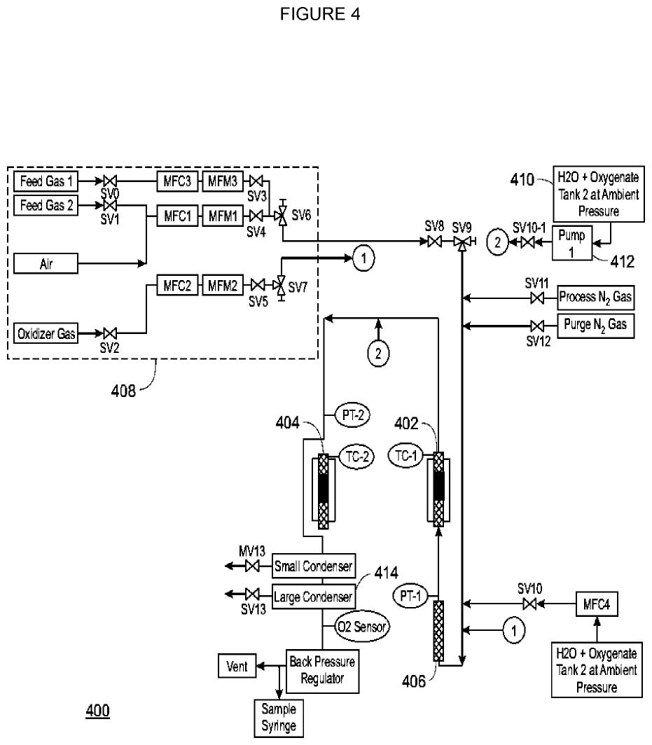

FIG. 4 is a simplified block flow diagram of a fixed bed reactor unit (FBRU) 400. The FBRU 400 was used to evaluate the use of a preheater.

The FBRU 400 consists of two fixed bed tubular reactors 402 and 404. Each reactor 402 and 404 is heated or cooled by a circulating closed loop oil bath which feeds into the jacket of the reactor. The reactors 402 and 404 are made out of 316 stainless steel and the reactor bed dimension is reported in Table 1. For the experiments described herein, only reactor 1 402 out of the two reactors was used, and reactor 2 404 was bypassed as shown in FIG. 4. A steam generator, used as a feed preheater 406 in examples herein, is placed prior to the reactor 1 402 to provide preheating and feed evaporation capability to the unit. The dimension of the feed preheater 406 is reported in Table 1 as well. To simplify the drawing, not every unit is labeled. In FIG. 4, PI stands for “pressure indicator”, SV stands for “solenoid valve”, MV stands for “manual valve”, MFC stands for “mass flow controller”, and MFM stands for “mass flow meter”. Generally, feed gas 1 and feed gas 2 may include any mixtures of methane and hydrogen.

Two feed gases can be fed through the reactors separately, for example, from a gas blending cabinet 408. For example, a first feed gas mixture is a mixture of oxygen-ethane-ethylene-carbon dioxide, in which the molar ratio of O₂ is controlled to remain below the flammability limit of the hydrocarbon mixture, which includes ethane and ethylene. The first feed gas can be used for conducting an ODH reaction or other related experiments, such as the empty reactor tube experiments described herein. The flow of gases is controlled by mass flow controllers (MFC). A second feed gas that may be provided from the gas blending cabinet 408 is air, which may be used for catalyst regeneration.

In addition to the feed gases from the gas blending cabinet 408, an oxygenate-water mixture 410 can be co-fed along with the mentioned feed gases into the inlet of the reactor 1 402 using a pump 412. In some implementations, a mass flow controller is used to feed an oxygenate-water mixture to the inlet of feed preheater 406 which subsequently goes to reactor 2 404. The oxygenate-water mixture will then evaporate at the inlet of the reactors. The oxygenate can be methanol, ethanol, acetic acid.

In may be noted that the preheater unit 406 and empty reactor unit 402 combined were used to mimic a preheater located upstream of an ODH reactor. The temperatures of the reactors 402 and 404 are monitored using 7-point thermocouples. If catalyst is loaded in the reactor, the catalyst bed is loaded such that at least 2 point of the thermocouples remain inside the catalyst bed. The arithmetic average of the thermocouple points inside the catalyst bed represents the average reactor temperature. If catalyst is not loaded in the reactor, for example, for empty reactor tube experiments, then the arithmetic average of the 7 thermocouple points represents the average reactor temperature. The temperature in each reactor 402 and 404 is monitored and controlled based on maximum value of the thermocouple point 1-7, using the oil baths which feed into each corresponding reactor jacket. The pressure inside the reactors 402 and 404 can be controlled and adjusted using a back pressure regulator (SV13), located downstream of the large condenser 414.

In the examples described herein, catalyst was not used in the FBRU 400 to allow the gas phase and piping catalyzed reactions to be tested. Therefore, the section in Table 1 pertaining to catalyst loading and parameters has been omitted.

TABLE 1
FBRU Dimension of Reactor 402 and Feed Preheater 406.

| Preheater Dimensions | | | Tubular Reactor Dimensions | | |
|---|---|---|---|---|---|
| Inside | | Inside | Inside | | Inside |
| Diameter | Height | Volume | Diameter | Height | Volume |
| (cm) | (cm) | (cm3) | (cm) | (cm) | (cm3) |
| 0.94 | 381 | 381 | 2.12 | 170 | 599 |

After all of the experiments in the FBRU 400 were completed, reactor 1 402 was opened and inspected which resulted in identifying (˜2 gram) of a fouling compound. The CHNO analysis for this compound is shown in Table 2. Based on this analysis result, it can be inferred that 28.4 wt. % of the sample is organic elements while the rest are inorganic elements. The dominant organic elements were found to be oxygen and carbon. For cases in which the percentage drop in O₂ dry mole fraction does not match the percentage increase (or % generated) in undesirable oxygenated byproducts (such as CO, CO₂, acetic acid), it can be assumed that the O₂ has gone into this collected solid fouling. To identify the bulk content of the inorganic components present in the fouling sample, ICP-MS analysis was conducted. Based on the ICP-MS result the top identified inorganic elements along with their mass fraction are sodium (8.0 wt. %), aluminum (5.0 wt. %), tellurium (3.2 wt. %), molybdenum (2.4 wt. %), and iron (2.2 wt. %).

TABLE 2
CHNO Results for the Fouling Sample
Collected from FBRU1 Reactor
CHNO Results

| | | | | |
|---|---|---|---|---|
| C | H | N | O | Total |

Mass Fraction (wt. %)

| | | | | |
|---|---|---|---|---|
| 9.92 | 0.58 | <0.04 | 17.90 | 28.44 |

Mole Fraction (mol %-normalize to 100)

| | | | | |
|---|---|---|---|---|
| 32.77 | 22.90 | <0.12 | 44.22 | 100.00 |

For the FBRU experiments, GC analyzers were used for identifying the gas product effluent and liquid product effluent. The GC analyzers have a general detection limit of 0.01%, and were calibrated at least once a month to ensure accuracy of the data. For experiments at which a detected compound was close to the detection limit (<0.1), the corresponding GC chromatogram was manually analyzed to determine if the chromatogram reflect noise pattern or a clear peak pattern. Only if the peak pattern was observed, then the value was accepted, otherwise it was assumed to be zero.

In all the examples in the FBRU 400, the ethane dry gas volume fraction increase is an artifact of consuming more O₂ compared to ethane on molar basis. This implies that the increase in dry volume fraction of ethane is not reflective of an increase in volume flow rate of this compound in the product stream. The consumption of ethane and O₂ was attributed to formation of undesirable liquid or gas by-products, such as CO₂ and acetic acid, and the solid fouling described above. Conversion of ethane and O₂ to the mentioned undesirable by-products can be explained based on the simplified bulk reaction 1 and 2 which confirms higher relative consumption of O₂ compared to ethane on molar basis.

C₂H₆+7/2 O₂→2 CO₂+3 H₂O Reaction 1

C₂H₆+3/2 O₂→CH₃COOH+H₂O Reaction 2

The CHNO analysis conducted on the solid fouling also confirms higher relative consumption of O₂ compared to ethane on molar basis

Further, in the FBRU 400 examples, the term “preheater residence time” has been used. The preheater residence time is the sum of the time the gas spends in the feed preheater 406 and reactor 1 402.

For all of the FBRU 400 experiments, it is speculated that formation of acetic acid and CO₂ and oxygenated solid fouling are likely due to combination of gas phase reactions and surface catalytic reaction over the interior surface of reactor 1 402 and the feed preheater 406, which is 316 stainless steel for the tube in reactor 1 402 and Hastelloy C-276 for the tube in the feed preheater 406.

### EXAMPLE 1

### CO₂—C₂H₆—O₂ Empty Tube Experiment at 300° C. and Preheater Residence Time of 13 sec (Calculated at STP)

In order to explore the presence of the any gas phase reaction or inside vessel tube catalyzed reaction when preheating the CO₂—C₂H₆—O₂ feed mixture, the feed preheater 406 and reactor 1 402 was operated at the reactor feed composition and operating condition reported in Table 3. The dry feed gas and product gas compositions are reported in Table 4. The liquid feed and product compositions are reported in Table 5.

From the experimental results shown in Tables 4-6, a number of observations were made at a total residence time of 13 secs and reaction temperature of 300° C. The ethane dry gas volume fraction increased in the product stream compared to the feed stream (0.55% absolute increase). The increase was assumed not to be representative of real increase, but an artifact of O₂/Ethane conversion to undesirable byproducts and solid fouling as described above. Further, the oxygen dry gas volume fraction decreased in the product stream compared to feed stream (1.61% absolute decrease), and the CO₂ dry gas volume fraction increased in the product stream compared to feed stream (1.05% absolute increase).

A trace amount of liquid product (0.6 mg/min) was observed, but was not a sufficient amount for liquid GC-FID analysis. However, the results seen in other examples (2-4) indicates that the liquid product may include acetic acid.

From the observed decreases in O₂ dry volume fraction, the increase in CO₂ dry volume fraction and the formation of oxygenated fouling, it may be inferred that at the total preheater residence time of 13 seconds and reactor temperature of 300° C., detectable thermal reaction occurred (leading into formation of traces CO₂ and the solid fouling) which suggest this feed mixture needs to be premixed at reactor temperature below 300° C. and a preheater residence time of less than about 13 seconds to avoid the loss of ethane/O₂ feed mixture to undesirable products/fouling.

TABLE 3
Reactor Feed Composition Ranges and Operating Condition
Ranges for Experiments Conducted in this Section

| Preheater | Reactor | Pre- | | Reaction | | | |
|---|---|---|---|---|---|---|---|
| Residence | Residence | heater | Reactor | Inlet | Feed Composition | | |
| Time | Time | Temp. | Temp. | Pressure | (Vol %)² | | |
| (sec)¹ | (sec)¹ | (° C.) | (° C.) | (psig) | C₂H₆ | O₂ | CO₂ |
| 4 | 9 | 247 | 300 | 62 | 19.6 | 9.7 | 70.7 |

¹reactor inside volume is 599 cm³, steam generator inside volume is 381 cm³ and total feed flow rate is 3873 cm³/min (STP).
²Other components that may be present in an ODH feed, such as water, acetic acid, and ethylene, were not present in these tests.

TABLE 4
Feed and Product Dry Gas Composition for
Experiments Conducted in this Section

| | Dry Gas Composition (Vol %) | | | | | | |
|---|---|---|---|---|---|---|---|
| Comment | C₂H₂ | C₂H₆ | C₂H₄ | CO | CO₂ | CH₄ | O₂ |
| Feed | 0.00 | 19.60 | 0.00 | 0.00 | 70.70 | 0.00 | 9.70 |
| Product | 0.00 | 20.15 | 0.00 | 0.00 | 71.75 | 0.00 | 8.09 |

The experimental data included an average of two runs.

TABLE 5
Feed and Product Liquid Composition for
Experiments Conducted in this Section

| | Liquid Composition (wt. %) | | | | | |
|---|---|---|---|---|---|---|
| | | | | | | Other |
| | | | | Acetic | | Oxy- |
| Comment | H₂O | Methanol | Ethanol | Acid | Acetone | genates |
| Feed | 0 | 0 | 0 | 0 | 0 | 0 |
| Product | not | not | not | not | not | not |
| | mea- | mea- | mea- | mea- | mea- | mea- |
| | sured | sured | sured | sured | sured | sured |

A trace amount of liquid product (~0.6 mg/min) was observed in the liquid product, but was not enough for liquid GC-FID analysis. Therefore, no liquid composition was not reported for the product stream.

### EXAMPLE 2

### H₂O—C₂H₆—O₂ Empty Tube Experiment at 300° C. and Preheater Residence Time of 13 sec (calculated at STP)

In order to explore the presence of any gas phase reaction or inside vessel tube catalyzed reaction when preheating an H₂O—C₂H₆—O₂ feed mixture to 300° C., the feed preheater 406 and reactor 1 402 were operated at the reactor feed composition and operating conditions reported in Table 6. The dry feed gas and product gas compositions are reported in Table 7. The liquid feed and product compositions are reported in Table 8.

From the experimental results, a number of observations were made for the total residence time of 13 secs and reaction temperature of 300° C. The ethane dry gas volume fraction increased in the product stream compared to feed stream (0.65% absolute increase). The increase was assumed to be an artifact of the conversion of O₂ and ethane conversion to undesirable byproducts and solid fouling. Oxygen dry gas volume fraction decreased in the product stream compared to feed stream (0.69% absolute decrease). Further, CO₂ was detected at 0.04 vol. % in the product stream. As no CO₂ was provided in the feed stream, this was a 0.04% absolute increase. Similarly, acetic acid was detected at a 4.11% liquid weight fraction in the product stream. As no acetic acid was provided in the product stream compared to feed stream, this was a 4.11% absolute increase.

From the observed decrease in O₂ dry volume fraction, the increase in CO₂ dry volume fraction, the increase in acetic acid weight fraction, and the formation of oxygenated fouling, it can be inferred that that detectable thermal reactions occurred at the total preheater residence time of 13 seconds and the reactor temperature of 300° C., leading to formation of acetic acid (dominant product), CO₂ (trace product) and the solid fouling. This suggests that the H₂O—C₂H₆—O₂ feed mixture needs to be premixed at reactor temperature below 300 ° C. and preheater residence time below 13 seconds to avoid the loss of the feed mixture to undesirable products and fouling.

TABLE 6
Reactor Feed Composition Ranges and Operating Condition
Ranges for Experiments Conducted in this Section.

| Preheater | Reactor | Pre- | | Reaction | | | |
|---|---|---|---|---|---|---|---|
| Residence | Residence | heater | Reactor | Inlet | Feed Composition | | |
| Time | Time | Temp. | Temp. | Pressure | (Vol %)² | | |
| (sec)¹ | (sec)¹ | (° C.) | (° C.) | (psig) | H₂O | C₂H₆ | O₂ |
| 4 | 9 | 247 | 300 | 60 | 70.7 | 19.6 | 9.7 |

¹reactor inside volume is 599 cm³, steam generator inside volume is 381 cm³ and total feed flow rate is 3873 cm³/min (STP).
²Other components that may be present in an ODH feed, such as CO2, acetic acid, and ethylene, were not present in this test.

TABLE 7
Feed and Product Dry Gas Composition for
Experiments Conducted in this Section.

| | Dry Gas Composition (Vol %) | | | | | | |
|---|---|---|---|---|---|---|---|
| Comment | C₂H₂ | C₂H₆ | C₂H₄ | CO | CO₂ | CH₄ | O₂ |
| Feed | 0.00 | 66.89 | 0.00 | 0.00 | 0.00 | 0.00 | 33.11 |
| Product | 0.00 | 67.54 | 0.00 | 0.00 | 0.04 | 0.00 | 32.42 |

TABLE 8
Feed and Product Liquid Composition for
Experiments Conducted in this Section.

| | Liquid Composition (wt. %) | | | | | |
|---|---|---|---|---|---|---|
| | | | | | | Other |
| | | | | Acetic | | Oxy- |
| Comment | H₂O | Methanol | Ethanol | Acid | Acetone | genates |
| Feed | 100 | 0 | 0 | 0 | 0 | 0 |
| Product | 95.89 | 0 | 0 | 4.11 | 0 | 0 |

### EXAMPLE 3

### H₂O—C₂H₆—O₂ Empty Tube Experiment at 250° C. and Preheater Residence Time of 13 Sec.

In order to explore the presence of any gas phase reaction or inside vessel tube catalyzed reaction when preheating an H₂O—C₂H₆—O₂ feed mixture to 300° C., the feed preheater 406 and reactor 1 402 were operated at the reactor feed composition and operating conditions reported in Table 9. The dry feed gas and product gas compositions are reported in Table 10. The liquid feed and product compositions are reported in Table 11.

From the experimental results, a number of observations were made at total residence time of 14 secs and reaction temperature of 250° C. The ethane dry gas volume fraction increased in the product stream compared to feed stream (3.57% absolute increase). The increase was assumed not to be representative of real increase due to being an artifact of O₂ and ethane conversion to undesirable byproducts and solid fouling.

The volume fraction of oxygen in the dry gas decreased in the product stream compared to feed stream (3.57% absolute decrease). However, CO₂ was not detected during this experiment, and may be assumed to not be present.

Similarly, acetic acid was detected at a 1.04% liquid weight fraction in the product stream. As no acetic acid was provided in the product stream compared to feed stream, this was a 1.04% absolute increase

From the observed decrease in the dry volume fraction of O₂, the increase in liquid weight fraction of acetic acid, and formation of oxygenated fouling (reported in note 1), it can be inferred that at the total preheater residence time of 13 seconds and the reactor temperature of 250° C., detectable thermal reaction occurred (leading into formation of acetic acid and the solid fouling) which suggest this feed mixture needs to be premixed at reactor temperature below 250° C. and preheater residence time below 13 seconds to avoid the loss of ethane/O₂ feed mixture to undesirable products/fouling.

Comparing the results of examples 2 and 3, it can be inferred that at lower reactor temperatures (250° C. versus 300° C.), the rate of the unwanted thermal reactions is decreased. This is evidenced by generation of less acetic acid and no CO₂ in the product stream of the experiment conducted at a reactor temperature of 250° C. compared to the results at a reactor temperature of 300° C. It should be noted, that in examples 2 and 3, all other reactor operating conditions, including the feed composition were the same, allowing the isolation of the effect of reactor temperature on rate of the mentioned unwanted thermal reaction. The reaction responsible for forming a solid oxygenated compound is a direct function of feed effluent adsorbing/chemisorbing on the surface of the catalyst. In general, the rate of adsorption/chemisorption increases as the reactor temperature decreases. This could lead into potentially higher concentration of solid oxygenated fouling. However, since we have not collected solid oxygenated fouling prior to each experiment, therefore this speculation cannot be validated or rejected.

TABLE 9
Reactor Feed Composition Ranges and Operating Condition
Ranges for Experiments Conducted in this Section.

| Preheater | Reactor | Pre- | | Reaction | | | |
|---|---|---|---|---|---|---|---|
| Residence | Residence | heater | Reactor | Inlet | Feed Composition | | |
| Time | Time | Temp. | Temp. | Pressure | (Vol %)² | | |
| (sec)¹ | (sec)¹ | (° C.) | (° C.) | (psig) | H₂O | C₂H₆ | O₂ |
| 4 | 9 | 247 | 250 | 60 | 70.7 | 19.6 | 9.7 |

¹reactor inside volume is 599 cm³, steam generator inside volume is 381 cm³ and total feed flow rate is 3873 cm³/min (STP)
²Other components that may be present in an ODH feed, such as CO₂, acetic acid, and ethylene, were not present in this test.

TABLE 10
Feed and Product Dry Gas Composition for
Experiments Conducted in this Section.

| | Dry Gas Composition (Vol %) | | | | | | |
|---|---|---|---|---|---|---|---|
| | C₂H₂ | C₂H₆ | C₂H₄ | CO | CO₂ | CH₄ | O₂ |
| Feed | 0.00 | 66.89 | 0.00 | 0.00 | 0.00 | 0.00 | 33.11 |
| Product | 0.00 | 70.47 | 0.00 | 0.00 | 0.00 | 0.00 | 29.53 |

TABLE 11
Feed and Product Liquid Composition for
Experiments Conducted in this Section.

| | Liquid Composition (wt. %) | | | | | |
|---|---|---|---|---|---|---|
| | | | | | | Other |
| | | | | Acetic | | Oxy- |
| Comment | H₂O | Methanol | Ethanol | Acid | Acetone | genates |
| Feed | 100 | 0 | 0 | 0 | 0 | 0 |
| Product | 98.96 | 0 | 0 | 1.04 | 0 | 0 |

### EXAMPLE 4

### Acetic Acid-H₂O—C₂H₆—O₂ Empty Tube Experiment at 300° C. and Preheater Residence Time of 13 Sec.

In order to explore the presence of any gas phase reaction or inside vessel tube catalyzed reaction when preheating an acetic acid-H₂O—C₂H₆—O₂ feed mixture to 300° C. the feed preheater 406 and reactor 1 402 were operated at the reactor feed composition and operating conditions reported in Table 12. The dry feed gas and product gas compositions are reported in Table 13. The liquid feed and product compositions are reported in Table 14.

From the experimental results, a number of observations were made at a total residence time of 13 secs and reaction temperature of 300° C. The ethane dry gas volume fraction increased in the product stream compared to feed stream (4.20% absolute increase). The increase was assumed to be an artifact of the conversion of O₂ and ethane conversion to undesirable byproducts and solid fouling. Oxygen dry gas volume fraction decreased in the product stream compared to feed stream (4.26% absolute decrease). Further, CO₂ was detected at 0.06 vol. % in the product stream. Dry gas volume fraction increased in the product stream compared to feed stream (0.06% absolute increase. As no CO₂ was provided in the feed stream, this was a 0.06% absolute increase. The liquid weight fraction of the acetic acid decreased in the product stream compared to feed stream (2.41% absolute decrease).

From the observed decrease in O₂ dry volume fraction, the increase in CO₂ dry volume fraction, the decrease in acetic acid weight fraction and formation of oxygenated fouling, it can be inferred that detectable thermal reactions at the occurred total preheater residence time of 13 seconds and the reactor temperature of 250° C., leading into formation of the solid fouling and trace amount of CO₂. This suggests that the acetic acid-H₂O—C₂H₆—O₂ feed mixture needs to be premixed at a reactor temperature below 300° C. and preheater residence time below 13 seconds to avoid the loss of the feed mixture to undesirable products and fouling.

Comparing the result of example 4 to example 2, it can be inferred that, in the presence of acetic acid in the feed mixture, unwanted thermal reactions responsible for generation of acetic acid from ethane and O₂ is suppressed as evidenced by decrease in the acetic acid weight fraction in the experiment containing acetic acid in the feed effluent and observed opposite trend for the experiment without acetic acid in the feed. Further, the decrease in O₂ volume fraction in this example (4.26 vol. % absolute drop) is substantially higher than the decrease in O₂ volume fraction in this example 2 (0.69 vol. % absolute drop). Since for the example 4, the higher O₂ decrease is not proportional to the generated carbon based byproducts in the gas product stream and liquid product stream, therefore it is concluded that the O₂ has converted into the solid oxygenated fouling. From this conclusion, it can be speculated that presence of acetic acid in the feed stream may have led into increase in the formation rate of the solid oxygenated fouling. In the two comparative experiments, the reactor operating conditions and feed composition remained nearly unchanged (less than 1 absolute difference for volume fraction of each feed compound). This allowed the study of the effect of acetic acid in the feed stream on the formation of the mentioned unwanted thermal reaction.

TABLE 12
Reactor Feed Composition Ranges and Operating Condition
Ranges for Experiments Conducted in this Section.

| Preheater | Reactor | | | Reaction | | | | |
|---|---|---|---|---|---|---|---|---|
| Residence | Residence | Preheater | Reactor | Inlet | Feed Composition | | | |
| Time | Time | Temp. | Temp. | Pressure | (Vol %)² | | | |
| (sec)¹ | (sec)¹ | (° C.) | (° C.) | (psig) | H₂O | CH₃COOH | C₂H₆ | O₂ |
| 4 | 9 | 247 | 300 | 62 | 69 | 2 | 20 | 9 |

¹reactor inside volume is 599 cm³, steam generator inside volume is 381 cm³ and total feed flow rate is 3873 cm³/min (STP)
²Other components that may be present in an ODH feed, such as ethylene, were not present in these tests.

TABLE 13
Feed and Product Dry Gas Composition for
Experiments Conducted in this Section.

| | Dry Gas Composition (Vol %) | | | | | | |
|---|---|---|---|---|---|---|---|
| Comment | C₂H₂ | C₂H₆ | C₂H₄ | CO | CO₂ | CH₄ | O₂ |
| Feed | 0.00 | 68.97 | 0.00 | 0.00 | 0.00 | 0.00 | 31.03 |
| Product | 0.00 | 73.17 | 0.00 | 0.00 | 0.06 | 0.00 | 26.77 |

TABLE 14
Feed and product liquid composition for
experiments conducted in this section

| | Liquid Composition (wt. %) | | | | | |
|---|---|---|---|---|---|---|
| | | | | | | Other |
| | | | | Acetic | | Oxy- |
| Comment | H₂O | Methanol | Ethanol | Acid | Acetone | genates |
| Feed | 87.12 | 0 | 0 | 12.88 | 0 | 0 |
| Product | 89.53 | 0 | 0 | 10.47 | 0 | 0 |

### EXAMPLE 5

### Scale-Up Reactor Examples

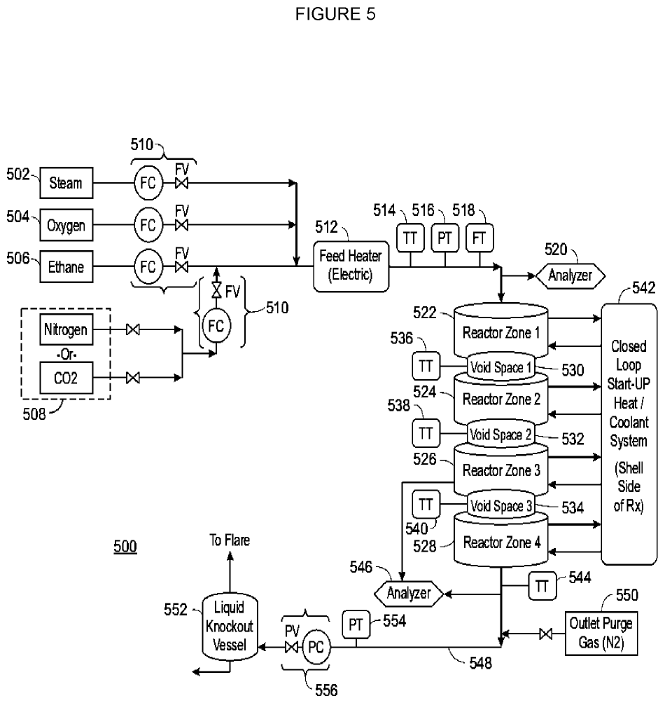

FIG. 5 is a simplified block flow diagram of a scale-up reactor system 500 used for larger scale tests. The scale up reactor system 500 has a number of feed streams, including, a steam feed stream 502, an oxygen feed stream 504, an ethane feed stream 506, and an inert gas feed stream 508. The inert gas feed stream 508 may include nitrogen, CO₂, or a mixture thereof. The supply for the steam feed stream 502 is at a pressure of 1170 kPag (kilopascals gauge) and at a temperature of 230° C. The supply for the oxygen feed stream 504 is at a pressure of 600 kPag and ambient temperature, with a 93 vol. % purity. The supply for the ethane feed stream 506 is at a pressure of 700 kPag and ambient temperature. The supply for the nitrogen feed stream is at a pressure of 680 kPag and ambient temperature. The supply for the CO2 feed stream is at a pressure of 700 kPag and ambient temperature.

As used herein, ambient temperature is the temperature of the atmosphere in the operational environment for the scale-up reactor system 500 without further heating or cooling. Generally, ambient temperature outdoors will vary depending upon the time of year, for example, from about −20° C. to about 25° C. However, the scale-up reactor system 500 is inside a test building, with a relatively stable ambient temperature of about 25° C. The ambient temperature may affect the molar amounts of the feeds added to the scale-up reactor system 500.

The addition of the each of the feed streams 502, 504, 506 and 508 is controlled by a flow controller system 510, which includes a flow controller (FC), for example, including a mass flow meter, and a flow control valve (FV). The feed streams 502, 504, 506 and 508 are blended upstream of a feed preheater 512. The feed preheater 512 is a 9 kW electric heater, with a maximum operating temperature of 375° C. A number of measurements are taken downstream of the feed preheater 512, for example, by a temperature transmitter (TT) 514, a pressure transmitter (PT) 516, a flow transmitter (FT) 518, and an analyzer 520. In some embodiments, the analyzer 520 is a gas chromatography (GC) system that may use a flame ionization detector (FID) detector, a thermal conductivity detector (TCD), a mass spectrometer, and the like. In some embodiments, the analyzer 520 incudes gas specific sensors, such as a CO₂ sensor, a CO sensor, and a light hydrocarbon sensor. In various embodiments, the analyzer 520 takes a regular sample from the flow from the feed preheater 512 and determines the levels of gases in the flow, including, for example, CO₂, CO, ethane, ethylene, O₂ and others.

The gas chromatography system used in the scale-up reactor system 500 was a Siemens Maxum Edition II Process Gas Chromatograph. A sample was analyzed every 150 seconds. Up to four different sample locations were routed to the same GC and analyzed in sequence However, the most common configuration had samples from two different locations alternating through the GC such that a new result was obtained for each sample location every 300 seconds. As used in these experiments, the gas chromatography system used a series of different columns in sequence, including a 5A mol sieve Hayesep, and a Shincarbon. A thermistor detector is used for the key components, such as CO₂, CO, oxygen and hydrocarbons, and a flame photometric detector for trace H2S.

The scale-up reactor system 500 includes four zones 522, 524, 526 and 528. The first two zones, zone 1 522 and zone 2 524 are about 6.7 L in volume, while the remaining zones, zone 3 526 and zone 4 528, are about 20.5 L in volume. Each of the zones 522, 524, 526 and 528 are separated by a void space 530, 532, and 534, respectively. The void spaces 530, 532 and 534 are each about 15 L in volume. Further, each of the void spaces 530, 532 and 534 are instrumented with a TT 536, 538 and 540 to measure the temperature of the material leaving the corresponding reactor zones 522, 524 and 526.

A temperature control system 542 is used to flow a temperature exchange media, SYLTHERM™ 800, through the shell side of the reactor zones 522, 524, 526 and 528. The temperature exchange media is used to control the temperature of the reactor zones by adding or removing heat. The maximum temperature of the SYLTHERM used for the scale-up reactor system 500 is 360° C.

A final TT 544 is located after the reactor zone 4 528. Further, a second analyzer 5466 is coupled into the outlet line 548 to determine the gas composition. The second analyzer 546 also has sample lines that can take samples from reactor zone 3 526 and reactor zone 4 528. The outlet line 548 is coupled to an outlet purge gas supply 550 to purge any materials from the outlet line 548 to a knockout vessel 552 for disposal. A pressure transmitter (PT) 554 is used with a pressure controller 556 to hold a back pressure on the reactor zone 4 528.

### Reactor Conditions for Tests

In the tests described herein, the reactor tubes in the first three reactor zones 522, 524 and 526 were filled with alumina catalyst support balls (⅛″ Christy Catalytics T99.5 PROX-SVERS) only. The reactor tubes were operated at a constant reactor outlet pressure (measured at PT 554) of 450 kPag throughout the test period. No heat was applied to the feed gas upstream of reactor zone 3 526. Accordingly, the feed gas temperature was at building ambient temperature throughout this period, which was about 25° C.).

The feed gas composition was held substantially constant throughout this period, albeit with a small amount of variation due to variations inherent in the mass flow control system, the composition of the ethane feed, and the oxygen supplied from the oxygen generator. Accordingly, the reactor feed stream include 86.5+/31 0.1 mol %, ethane, 12.5 mol % oxygen, 0.8 mol % argon, and 0.2+/−0.1 mol % ethylene.

### Scale-Up Reactor Example 1

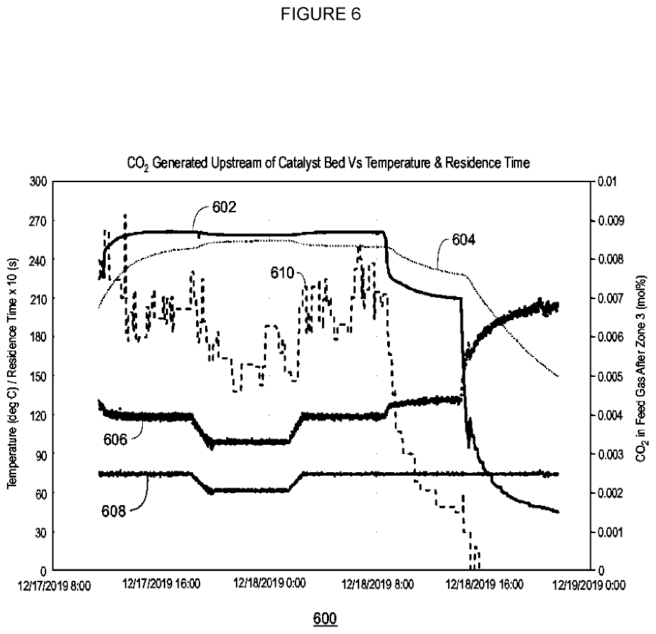

FIG. 6 is a plot 600 of the operating results for a first example using the scale-up reactor. During the tests, the reactor feeds were varied. Between December 17 @ 09:00 hours and December 17 @ 18:20 hours, the flow rate of feed gas was held constant at 9.33 kg/h. Between December 17 @ 20:00 hours and December 18 @ 01:30 hours, the flow rate of feed gas was held constant at 11.2 kg/h. Between December 18 @ 02:30 hours and December 18 @ 21:30 hours, the flow rate of feed gas was held constant at 9.33 kg/h.

The temperature of reactor zone 3 526 was temporarily adjusted by operating with SYLTHERM flowing through the shell-side in order to increase the process gas temperature. The SYLTHERM temperature 602 was measured from at the temperature control system 542. In this example, the reactor zone 3 526 mimics a preheater prior to a main ODH reactor. Between December 17 @ 09:00 hours and December 18 @ 08:30 hours, the flow rate of SYLTHERM was held constant at 100 kg/h. Between December 18 @ 08:50 hours and December 18 @ 14:15 hours, the flow rate of SYLTHERM was held constant at 50 kg/h. As of December 18, 2019 @ 14:18 hours, no SYLTHERM was flowing. The measured temperature 604 at the TT 540 in the void space 3 534 was skewed between December 17 @ 11:00 hours and December 17 @ 18:00 hours, due to the effect of heat absorption by the metal of the reactor shell and tubes. Until the system reached equilibrium at approximately 18:00 hours, the gas temperature leaving the tubes of reactor zone 3 526 was higher than the measured temperature 604 of void space 3 534.

The residence time in reactor zone 3 526 was calculated in two different ways, using a multiplier of ten in order to improve the resolution in the plot 600. The residence time 606 based on estimated gas density from regressed plot of density vs temperature & pressure generated by Aspen Properties for the feed gas mixture indicated above and assuming a void fraction of 0.4 in the tube volume. The equivalent residence time 608 is calculated assuming the feed gas was at STP conditions [21° C., 100 kPa].

The second analyzer 546 monitored the gas composition leaving reactor zone 3 526 throughout the test period. The ability to replicate similar results, for example, the trend 610 of CO₂, confirmed that the observed presence of trace amounts of CO₂ is not due to GC error and is a real positive value.

It can be noted that CO₂ was the only generated gas product observed in the effluent stream from reactor zone 3 526. Further, the CO₂ is only present in the effluent stream from reactor zone 3 526 at detectable concentrations when the SYLTHERM is flowing through the shell-side of the reactor, raising the process gas temperature above 210° C. Once the SYLTHERM is removed, the CO₂ present in the process gas drops to non-detectable amounts, as shown by the trend 610.

Oxygenates, such as acetic acid, were not observed in this example, but the apparatus did not allow for the collection of liquid samples. Liquids can only be collected from the GC sample conditioning system of the second analyzer 546. However, there was not enough liquid present at the liquid knockout upstream of the analyzer to obtain a sample. Thus, oxygenates may have been present in the process stream at the outlet of reactor zone 3 528, but they could not be detected by the GC. It can be inferred from the results of this example that to minimize unwanted reactions in the preheater, the preheater temperature should remain below about 210° C. and feed gas residence time should be minimized.

### Scale-Up Reactor Example 2

In this example, the feed preheater 512 was used to heat the feed gas prior to the gas coming into contact with the catalyst. The feed preheater 512 is made from Hastelloy C-276, and has a shell of 3″ schedule 40 pipe and a length of about 2.813 m giving an internal volume of 11.57 L, accounting for displacement of the heating element. The feed gas flows through inside volume of the shell. The heating element is located inside the shell and comes in direct contact with the feed gas flow.

Using the feed preheater 512, an experiment was conducted in January 2020 to determine if the earlier observations of CO₂ generation in the absence of catalyst could be replicated. The earlier example is described herein as scale-up reactor example 1.

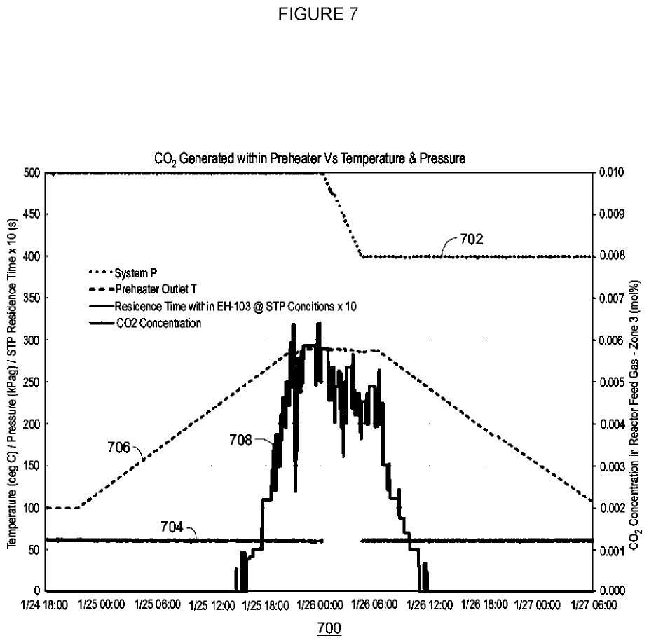

FIG. 7 is a plot 700 of the results of the second example using the scale-up reactor. In this example, the outlet of reactor zone 3 526 outlet was sampled every 5 minutes by the second analyzer 546, using a GC system to monitor the gas composition. As described with respect to scale-up reactor example 1, the reactor tubes in the first three reactor zones, 522, 524 and 526, were filled with alumina catalyst support balls (⅛″ Christy Catalytics T99.5 PROX-SVERS) only. The feed gas did not come into contact with active catalyst between the outlet of the feed preheater 512 and the online GC sampling location.

The scale-up reactor system 500 was operated at a controlled system pressure 702 of 500 kPag for the first 30 hours of the experiment (January 24 @ 18:00 to January 26 @ 00:01). A 4.5-hour transition period (from 00:01 hours to 04:30 hours. on January 26) was used to reduce the system pressure from 500 kPag to 400 kPag. For the final 25.5 hours of the experiment (January 26 @ 04:30 hours to January 27 @ 06:00 hours), the process was operated at a controlled system pressure of 400 kPag. No heat was applied to the feed gas within the reactor zones 1 522, 2 524 or 3 526.

The feed rate to the scale-up reactor system 500 was controlled throughout the experiment. Between January 24 @ 18:00 hours and January 26 @ 00:01 hours, the flow rate of feed gas was held constant at 8.2 kg/h. However, between January 26 @ 00:01 hours and 04:30 hours, the flow rate and composition of feed gas was not held constant. Oxygen flow was partially disrupted during the transition in system pressure. Between January 26 @ 04:30 hours and January 27 @ 06:00 hours, the flow rate of feed gas was held constant at 8.2 kg/h.

Outside of the pressure transition period on January 26 between 00:01 hours and 04:30 hours, the composition of the feed gas to the scale-up reactor system 500 was held substantially constant throughout this period, albeit with a small amount of variation due to variations inherent in the mass flow control system, the composition of the ethane feed, and the oxygen supplied from the oxygen generator. Accordingly, the reactor feed stream included 47.8 mol %, ethane, 10.5 mol % oxygen, 0.6 mol % argon, and 0.3 mol % ethylene. Reactor zones 1 522, 2 524, and 3 526 had no flow of SYLTHERM through the shell-side which implies that the reactor temperature inside these zones are likely close to ambient temperature.

The residence time 704 of the gas mixture, assuming it remained at STP conditions (21° C., 100 kPa) throughout, was calculated to remain between 6.0 and 6.2 s. However, it did not remain exactly constant due to process variations. The actual residence time of the gas within the feed preheater 512 could not be calculated as the inlet temperature was not measured.

As shown in the plot 700, the outlet temperature 706 of the preheater was ramped from about 100° C. to about 275° C. and back down to 100° C. CO₂ 708 is only present in the reactor zone 3 526 outlet gas at detectable concentrations when the process gas temperature at the outlet of the feed preheater 512 is above 260° C. The generation of the CO₂ was reduced at lower system pressure and lower residence time.

In scale-up reactor example 2, the feed preheater 512 mimics a preheater before a main ODH reactor. In scale-up reactor example 1, the reactor zone 3 526 mimics a preheater prior to the main ODH reactor. Comparing the results in example 2 to example 1, it can be inferred that at relatively same STP resident time, about 6-7 seconds, and similar preheater temperature, about 260° C.-290° C. The different feed composition and absence of support alumina in the preheater did not appear to impact the quantity of detected CO₂ at the outlet of the preheater, which was in the range of about 0.002-0.007 mol %). As for scale-up reactor example 1, oxygenates, such as acetic acid, were not observed in this example. However, the apparatus did not allow for the collection of liquid samples.

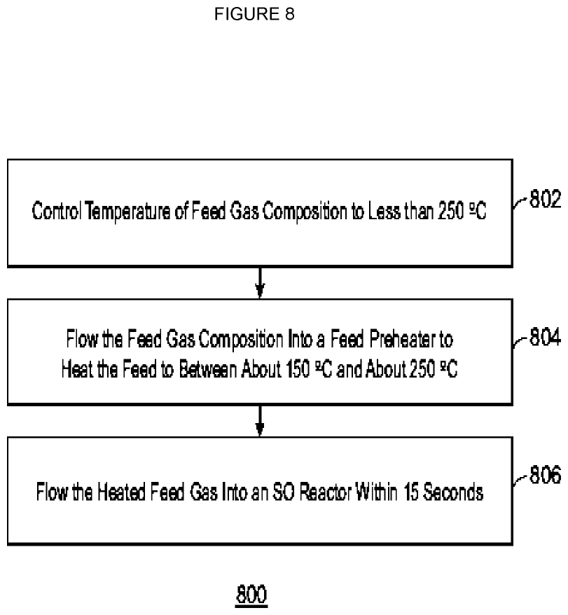

FIG. 8 is a process flow diagram of a method 800 for increasing the yield from an ODH reactor while decreasing side products and fouling upstream of the ODH reactor. The method 800 is based on the examples described herein. The method begins at block 802, when the temperature of a feed gas composition is controlled to be less than about 250° C. For example, if steam is used to dilute a feed of ethane and oxygen, the temperature is controlled at less than about 250° C., less than about 225° C., or less than 200° C., or lower. At block 804, the feed gas composition is flowed into a feed preheater to heat the feed to a maximum temperature of between about 150° C. and about 250° C. The feed preheater may be attached to, or incorporated in, the ODH reactor to decrease the amount of residence time that the feed is at the maximum temperature. At block 806, the heated feed gas is flowed into an ODH reactor within about 15 seconds, or less, of reaching the maximum temperature.

An embodiment described herein provides a method for increasing a yield from an oxidative dehydrogenation (ODH) reactor. The method includes controlling a temperature of a feed gas composition at less than 250° C. The feed gas composition is flowed through a feed preheater to form a heated feed gas, wherein in the feed preheater the feed gas composition is heated to between 150° C. and 250° C. The heated feed gas is flowed into an ODH reactor less than 15 seconds after leaving the feed preheater.

In an aspect, the method includes heating the feed preheater with heat from the ODH reactor. In an aspect, the method includes flowing the feed gas composition into tubing within the ODH reactor as the feed preheater.

In an aspect, the method includes controlling a residence time and the temperature of the feed gas composition by selecting a length of the tubing. In an aspect, the method includes controlling a residence time and the temperature of the feed gas composition by selecting a length of piping coupling the preheater to the reactor. In an aspect, the method includes controlling the temperature of the feed gas composition to less than 200° C. prior to flowing the feed gas composition through the feed preheater.

In an aspect, the method includes flowing the heated feed gas into the ODH reactor within 13 seconds of reaching a maximum temperature. In an aspect, the method includes including forming the feed gas composition by blending steam, a light hydrocarbon, and oxygen in a flooded blending tank.

In an aspect, the method includes controlling a temperature of an interstage feed stream at less than 250° C., flowing the interstage feed stream through an interstage feed preheater to form a heated interstage feed stream, wherein the interstage feed stream is heated to between 150° C. and 250° C. The heated interstage feed stream is flowed into the ODH reactor in less than 13 seconds after the interstage feed stream reaches a maximum temperature.

In an aspect, the method includes forming the interstage feed stream in a second ODH reactor. In an aspect, the method includes cooling an effluent from the second ODH reactor in an effluent cooler.

Another embodiment described herein provides a feed preheater for an ODH reactor, wherein the feed preheater is configured to heat a reactor feed to between about 150° C. and about 250° C. and flow the reactor feed into the ODH reactor within 15 seconds of the reactor feed reaching a maximum temperature.

In an aspect, the feed preheater is attached to the ODH reactor. In an aspect, the feed preheater is configured to be heated by excess heat from the ODH reactor. In an aspect, the feed preheater includes tubing incorporated into the ODH reactor. In an aspect, a length of the tubing incorporated into the ODH reactor is selected to adjust a residence time and a temperature of the reactor feed. In an aspect, the feed preheater is configured to flow the reactor feed into the ODH reactor within 13 seconds of the reactor feed reaching a maximum temperature.

In an aspect, the feed preheater includes effluent piping selected in length to flow of the reactor feed into the ODH reactor within 15 seconds of the reactor feed reaching a maximum temperature.

Another embodiment described herein provides an oxidative dehydrogenation (ODH) reactor including a feed preheater, wherein the feed preheater is configured to heat a reactor feed to between about 150° C. and about 250° C., and to introduce the reactor feed into the ODH reactor within less than 15 seconds of the reactor feed reaching a maximum temperature.

In an aspect, the ODH reactor includes a feed tube disposed within the ODH reactor, wherein the feed tube is heated by contents of the ODH reactor, and a length of the feed tube is selected to heat the reactor feed to between about 150° C. and about 250° C. In an aspect, the ODH reactor includes a second feed preheater for heating a second reactor feed to between about 150° C. and about 250° C., and to introduce the second reactor feed into the ODH reactor within less than 15 seconds of the reactor feed reaching a maximum temperature. In an aspect, the second reactor feed includes acetic acid, ethanol, or a recycle stream, or any combinations thereof.

A number of implementations have been described. Nevertheless, it will be understood that various modifications may be made without departing from the spirit and scope of the disclosure.

### INDUSTRIAL APPLICABILITY

The present disclosure relates to a method and system for increasing yields from an oxidative dehydrogenation reactor.

1. A method, comprising:
controlling a temperature of a feed gas composition to less than 250° C.;
flowing the feed gas composition through a feed preheater to form a heated feed gas, wherein in the feed preheater the feed gas composition is heated to between 150° C. and 250° C.; and
flowing the heated feed gas into an oxidative dehydrogenation (ODH) reactor less than 15 seconds after leaving the feed preheater.

2. The method of claim 1, further comprising heating the feed preheater with heat from the ODH reactor.

3. The method of claim 1, wherein the ODH comprises tubing that serves as the feed preheater, and the method comprises flowing the feed gas composition into tubing.

4. The method of claim 3, further comprising controlling a residence time and the temperature of the feed gas composition in the tubing by selecting a length of the tubing.

5. The method of claim 1, wherein piping couples the preheater and the ODH reactor, and the method further comprises controlling a residence time and the temperature of the feed gas composition by selecting a length of the piping.

6. The method of claim 1, further comprising controlling the temperature of the feed gas composition to less than 200° C. prior to flowing the feed gas composition through the feed preheater.

7. The method of claim 1, further comprising flowing the heated feed gas into the ODH reactor within 13 seconds of reaching a temperature of less than 250° C.

8. The method of claim 1, further comprising forming the feed gas composition by blending steam, a light hydrocarbon, and oxygen in a flooded blending tank.

9. The method of claim 1, further comprising:
controlling a temperature of an interstage feed stream at less than 250° C.;
flowing the interstage feed stream through an interstage feed preheater to form a heated interstage feed stream, wherein the interstage feed stream is heated to between 150° C. and 250° C.; and
injecting the heated interstage feed stream into the ODH reactor less than 13 seconds after the interstage feed stream is heated.

10. The method of claim 9, further comprising forming the interstage feed stream in a second ODH reactor.

11. The method of claim 10, further comprising cooling an effluent from the second ODH reactor in an effluent cooler.

12. A feed preheater for an oxidative dehydrogenation (ODH) reactor, wherein the feed preheater is configured to heat a reactor feed to between about 150° C. and about 250° C. and flow the reactor feed into the ODH reactor within 15 seconds of the reactor feed reaching a maximum temperature.

13. The feed preheater of claim 12, wherein the feed preheater is attached to the ODH reactor.

14. The feed preheater of claim 13, wherein the feed preheater is configured to be heated by excess heat from the ODH reactor.

15. The feed preheater of claim 12, wherein the feed preheater comprises tubing incorporated into the ODH reactor.

16. The feed preheater of claim 15, wherein a length of the tubing incorporated into the ODH reactor is configured to adjust a residence time and a temperature of the reactor feed.

17. The feed preheater of claim 12, wherein the feed preheater is configured to flow the reactor feed into the ODH reactor within 13 seconds of the reactor feed reaching a maximum temperature.

18. The feed preheater of claim 12, further comprising effluent piping having a length configured to flow the reactor feed into the ODH reactor within 15 seconds of the reactor feed reaching a maximum temperature.

19. An oxidative dehydrogenation (ODH) reactor, comprising:
a feed preheater,
wherein the feed preheater is configured to heat a reactor feed to between about 150° C. and about 250° C., and the feed preheater is configured to introduce the reactor feed into the ODH reactor within less than 15 seconds of the reactor feed reaching a maximum temperature.

20. The ODH reactor of claim 19, further comprising a feed tube disposed within the ODH reactor, wherein the feed tube is configured to be heated by contents of the ODH reactor, and a length of the feed tube is configured to heat the reactor feed to between about 150° C. and about 250° C.

21.-22. (canceled)
